SEQUENCE LISTING (9 sequences)

SEQ ID NO: 1 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 2 - protein, 49 aa, Homo sapiens
AMVSEFLKQAWFIENEEQEYVQTVKSSKGGPGSAVSPYPTFNPSSDVAA

SEQ ID NO: 3 - protein, 47 aa, Homo sapiens
AMVSEFLKQAWFIENEEQEYVQTVKSSKGGPGSAVSPYPTFNPSSDV

SEQ ID NO: 4 - protein, 45 aa, Homo sapiens
AMVSEFLKQAWFIENEEQEYVQTVKSSKGGPGSAVSPYPTFNPSS

SEQ ID NO: 5 - protein, 49 aa, synthetic construct
  REGION 1..49: note = Mutated Annexin A1 2-50 V24L
  SITE 24: note = MISC_FEATURE - Amino acid V substituted with L
AMVSEFLKQAWFIENEEQEYVQTLKSSKGGPGSAVSPYPTFNPSSDVAA

SEQ ID NO: 6 - protein, 47 aa, synthetic construct
  REGION 1..47: note = Mutated Annexin A1 2-48 V24L
  SITE 24: note = MISC_FEATURE - Amino acid V substituted with L
AMVSEFLKQAWFIENEEQEYVQTLKSSKGGPGSAVSPYPTFNPSSDV

SEQ ID NO: 7 - protein, 45 aa, synthetic construct
  REGION 1..45: note = Mutated Annexin A1 2-46 V24L
  SITE 24: note = MISC_FEATURE - Amino acid V substituted with L
AMVSEFLKQAWFIENEEQEYVQTLKSSKGGPGSAVSPYPTFNPSS

SEQ ID NO: 8 - protein, 25 aa, Homo sapiens
AMVSEFLKQAWFIENEEQEYVQTVK

SEQ ID NO: 9 - protein, 25 aa, synthetic construct
  REGION 1..25: note = Mutated Annexin A1 2-26 V24L
  SITE 24: note = MISC_FEATURE - Amino acid V substituted with L
AMVSEFLKQAWFIENEEQEYVQTLK

(19) United States
(12) Patent Application Publication
(10) Pub. No.: US 2026/0263555 A1
(43) Pub. Date: Sep. 10, 2026

(54) ANNEXIN A1 DELAYED DOSING

(71) Applicant: Reso Ther Pharma A/S, Holte (DK)

(72) Inventors: Thomas Engelbrecht Nordkild Jonassen, Holte (DK); Samra Joke Sanni, Frederiksberg (DK)

(21) Appl. No.: 19/163,901

(22) PCT Filed: Mar. 13, 2024
(86) PCT No.: PCT/EP2024/056606

§ 371 Date: Sep. 10, 2025

(30) Foreign Application Priority Data
Mar. 13, 2023 (EP) 23161538.6

(51) Int. Cl. A61K 38/17; A61K 47/26; A61P 9/10

(52) U.S. Cl. CPC A61K 38/1709; A61K 47/26; A61P 9/10

(57) ABSTRACT

The present disclosure relates to an Annexin A1 (AnxA1) N-terminal-peptide and pharmaceutical compositions comprising an Annexin A1 (AnxA1) N-terminal-peptide for use in the treatment of an ischemic condition and/or a reperfusion injury.

### TECHNICAL FIELD

The present disclosure relates to an Annexin A1 (AnxA1) N-terminal-peptide and pharmaceutical compositions comprising the Annexin A1 (AnxA1) N-terminal-peptide for use in the treatment of an ischemic condition and/or a reperfusion injury, wherein a first dosage of the AnxA1 N-terminal-peptide is administered to a human subject more than 1 hour post establishment of reperfusion.

### BACKGROUND

The Annexin super-family consists of 13 calcium phospholipid binding proteins with significant biological and structural homology. Annexins are structurally divided into a highly conserved core domain and a variable N-terminal domain. Annexin A1 (ANXA1, 37 kDa—SEQ ID NO:1) is an anti-inflammatory protein that inhibits extravasation of blood-borne polymorphonuclear leukocyte (PMN) into the surrounding tissue. The protein binds to the N-formyl peptide receptor (FPR) 2 or FPR-L1 receptor, where it initiates a cascade of signaling events. Following an inflammatory stimulus, migration of blood-borne polymorphonuclear leukocyte (PMN) into the surrounding tissue takes place. Transmigration or extravasation of PMN is regulated by mediators such as adhesion molecules, cytokines and proteases, which control the pro-inflammatory and anti-inflammatory processes. The disruptive potential of the PMN is high and potentially self-damaging. Thus, controlling extravasation of PMN and the inflammatory response is important.

For therapeutic purposes as an anti-inflammatory agent, the full Annexin A1 protein has numerous disadvantages relative to functional fragments or modified versions thereof. The large size of the protein makes it more difficult to deliver by techniques that are possible with a smaller polypeptide (e.g. transdermally or transmucosally). For use to treat inflammation of the eyes, a smaller molecule is expected to be better able to penetrate the corneal epithelium. Also, susceptibility to proteolytic degradation is a particular concern for all peptide pharmaceuticals, especially large ones and especially if oral delivery (preferred by many patients) is contemplated.

Some Annexin A1 derivatives lacking significant regions on the N-terminal side of the polypeptide have been shown to lack significant activity in some assays of inflammation and mediator release, whereas the full length N-terminus N-acetyl Annexin A1 was deemed biologically active in several systems. A number of peptides primarily derived from the unique N-terminal portion of the Annexin A1 protein have been shown to possess anti-inflammatory properties. One of the most extensively studied Annexin A1 peptides is peptide Ac2-26, which mimics the 2^{nd} to the 26^{th} amino acids of the 54-amino acid N-terminal region. It has been shown that Annexin A1 and its N-terminal peptide (Ac2-26) exert the majority of their anti-inflammatory action through the FPR2/Lipoxin A4 (FPR2/Alx) receptor. In vivo the Ac2-26 peptide has been shown to exert an anti-inflammatory effect in models of myocardial ischaemia reperfusion (I/R), mesentery I/R, glycogen peritonitis and IL1 air pouch, where it was reported to significantly reduce the recruitment of neutrophils to the site of injury/inflammation.

Shorter versions of the Ac2-26 peptide, such as peptides Ac2-12 and Ac2-6, have also been shown to elicit some degree of anti-inflammatory effects in acute models of inflammation. Longer polypeptides with anti-inflammatory effects, such as polypeptides corresponding to amino acid residues 2-48, 2-50 and 11-48, have been disclosed, including protease-resistant V24L variants (WO 2012/174397 and WO 2022/038281).

A myocardial infarction, commonly known as a heart attack, occurs when blood flow decreases or stops to the coronary artery of the heart, causing damage to the heart muscle. Further tissue damage in the form of reperfusion injury may occur upon reperfusion, i.e., when blood supply returns to the heart after a period of ischemia or lack of oxygen. As the intervention of reperfusion in myocardial infarct patient often includes reperfusion of more than one vessel, there is a need for developing effective treatments that do not require administration of a drug immediately after reperfusion of the first vessel, but wherein administration of the drug can be delayed for a certain period of time after reperfusion of the ischemic myocardium has be established.

### SUMMARY

There is a need for developing safe and effective treatments of an ischemic condition and/or a reperfusion injury, wherein administration of a first dosage of a drug can be delayed for a certain time post establishment of reperfusion. The present inventors show that administration of an AnxA1 N-terminal-peptide significantly reduced infarct size when administered 3 times, with the first dosage given at establishment of reperfusion (reperfusion start). Surprisingly, delaying the treatment with the AnxA1 N-terminal-peptide for up to two hours post establishment of reperfusion reduced infarct size to a similar degree as when the AnxA1 N-terminal-peptide was administered at establishment of reperfusion (reperfusion start). Thus, delaying the AnxA1 N-terminal-peptide administration for up to two hours post establishment of reperfusion does not compromise the cardioprotective effects of the AnxA1 N-terminal-peptide.

In some embodiments, the present disclosure relates to an Annexin A1 (AnxA1) N-terminal-peptide for use in the treatment of an ischemic condition and/or a reperfusion injury, wherein a first dosage of the AnxA1 N-terminal-peptide is administered to a human subject more than 1 hour post establishment of reperfusion.

In some embodiments, the present disclosure relates to a pharmaceutical composition comprising an Annexin A1 (AnxA1) N-terminal-peptide and at least one pharmaceutically acceptable excipient for use in the treatment of an ischemic condition and/or a reperfusion injury, wherein a first dosage of the AnxA1 N-terminal-peptide is administered to a human subject more than 1 hour post establishment of reperfusion.

### DESCRIPTION OF DRAWINGS

FIG. 1 shows the effect of delayed Annexin A1 peptide dosing in a rat model of acute myocardial infarction. FIG. 1A shows treatment regime of Group 1, 2 and 3. FIG. 1B shows that administration of Peptide 1 significantly reduced infarct size, both when the first dosage was given at establishment of reperfusion (reperfusion start) T=0 (Group 1) when the first dosage was given at one hour post establishment of reperfusion T=1 (Group 2) and when the first dosage was given at two hours post establishment of reperfusion T=2 (Group 3). *p<0.05: One-way Anova vs vehicle. #p<0.05, ##p<0.01: t-test between each treatment group versus vehicle

FIG. 2 shows the effect of Peptide 1 treatment for 1 day and 3 days on cardiac function in a rat model of acute myocardial infarction. FIG. 2 shows that administration of at least three dosages of Peptide 1 significantly improves long-term cardiac function. *p<0.05: One-way Anova vs vehicle.

### DEFINITIONS

The term “a” denotes at least one, i.e., one or more.

The terms “approximately” and “about” as referred herein are synonymous. In some embodiments, “approximately” and “about” refer to the recited amount, value, or duration±10%, ±9%, ±8%, ±7%, ±6%, ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5%±0.4%, ±0.3%, ±0.2% or ±0.1%. In some embodiments, “approximately” and “about” refer to the listed amount, value, or duration±10%, ±5%, ±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration±10%. In some embodiments, “approximately” and “about” refer to the listed amount, value, or duration±5%. In some embodiments, “approximately” and “about” refer to the listed amount, value, or duration±1%.

The term “Dosing interval” as used herein refers to the time period between administration of two dosages.

The term “Administration time” as used herein refers to the time where an administration or infusion, such as an i.v. infusions, starts.

A pharmaceutically acceptable excipient is understood as any excipient acceptable to include in a pharmaceutical composition. Pharmaceutically acceptable excipients include, but are not restricted to, bulking agents, tonicity agents, isotonicity modifiers, stabilisers, surfactants, buffers, water, carbohydrates and sugar alcohols.

An intravenous infusion is the direct injection of a fluid medication into a vein through an intravenous line, needle, cannula or catheter. Administration of drugs via IV infusion is usually necessary when longer or continuous systemic exposures are required to elicit a therapeutic effect. Unlike injection, infusion often uses a pump or the natural force of gravity to deliver fluids into the body.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The terms “treatment” and “treating” as used herein refer to the management and care of a patient for the purpose of combating a condition, disease or disorder. The term is intended to include the full spectrum of treatments for a given condition from which the patient is suffering. The patient to be treated is preferably a mammal, in particular a human being. The patients to be treated can be of various ages.

Reperfusion refers to restoration of blood flow to a previously ischemic tissue or organ.

Reperfusion injury, sometimes called ischemia-reperfusion injury (IRI) or reoxygenation injury, is the tissue damage caused when blood supply returns to tissue after a period of ischemia or lack of oxygen (anoxia or hypoxia).

The term “Establishment of reperfusion” as used herein refers to the time point where blood flow to an organ or tissue is restored after having been blocked. This might also be referred to as “Reperfusion start”.

The term “Post establishment of reperfusion” as used herein refers to a time point occurring after reperfusion has been established. This might also be referred to as “Post reperfusion start”.

### DETAILED DESCRIPTION

It is an aspect of the present disclosure to provide an Annexin A1 (AnxA1) N-terminal-peptide for use in the treatment of an ischemic condition and/or a reperfusion injury, wherein a first dosage of the AnxA1 N-terminal-peptide is administered to a human subject more than 1 hour post establishment of reperfusion.

It is also an aspect of the present disclosure to provide a pharmaceutical composition comprising an Annexin A1 (AnxA1) N-terminal-peptide and at least one pharmaceutically acceptable excipient for use in the treatment of an ischemic condition and/or a reperfusion injury, wherein a first dosage of the AnxA1 N-terminal-peptide is administered to a human subject more than 1 hour post establishment of reperfusion.

It is an aspect of the present disclosure to provide an Annexin A1 (AnxA1) N-terminal-peptide for use in the treatment of an ischemic condition and/or a reperfusion injury, wherein a first dosage of the AnxA1 N-terminal-peptide is to be administered to a human subject more than 1 hour post establishment of reperfusion.

It is also an aspect of the present disclosure to provide a pharmaceutical composition comprising an Annexin A1 (AnxA1) N-terminal-peptide and at least one pharmaceutically acceptable excipient for use in the treatment of an ischemic condition and/or a reperfusion injury, wherein a first dosage of the AnxA1 N-terminal-peptide is to be administered to a human subject more than 1 hour post establishment of reperfusion.

It is also an aspect of the present disclosure to provide a method of treating an ischemic condition and/or a reperfusion injury, the method comprising administration of a first dosage of an Annexin A1 (AnxA1) N-terminal-peptide to a human subject more than 1 hour post establishment of reperfusion.

It is also an aspect of the present disclosure to provide a method of treating an ischemic condition and/or a reperfusion injury, the method comprising administration of a pharmaceutical composition comprising an Annexin A1 (AnxA1) N-terminal-peptide and at least one pharmaceutically acceptable excipient to a human subject, wherein a first dosage of the AnxA1 N-terminal-peptide is administered to the human subject more than 1 hour post establishment of reperfusion.

It is also an aspect of the present disclosure to provide a method of reducing necrosis caused by an ischemic condition and/or a reperfusion injury, the method comprising administration of a first dosage of an Annexin A1 (AnxA1) N-terminal-peptide to a human subject more than 1 hour post establishment of reperfusion.

It is also an aspect of the present disclosure to provide a method of reducing infarct size of an infarct caused by an ischemic condition and/or a reperfusion injury, the method comprising administration of a first dosage of an Annexin A1 (AnxA1) N-terminal-peptide to a human subject more than 1 hour post establishment of reperfusion.

It is also an aspect of the present disclosure to provide a method of reducing infarct size of a myocardial infarct caused by an ischemic condition and/or a reperfusion injury, the method comprising administration of a first dosage of an Annexin A1 (AnxA1) N-terminal-peptide to a human subject more than 1 hour post establishment of reperfusion.

In some embodiments, the present disclosure relates to the use of an Annexin A1 (AnxA1) N-terminal-peptide for the manufacture of a medicament for use in the treatment of an ischemic condition and/or an inflammatory condition, wherein a first dosage of the AnxA1 N-terminal-peptide is to be administered to a human subject more than 1 hour post establishment of reperfusion.

In some embodiments, the ischemic condition is followed by reperfusion.

In some embodiments, the present disclosure relates to an Annexin A1 (AnxA1) N-terminal-peptide for use in the treatment of an ischemic condition and/or a reperfusion injury, wherein a first dosage of the AnxA1 N-terminal-peptide is administered to a human subject more than 1 hour post establishment of reperfusion.

In some embodiments, the present disclosure relates to a pharmaceutical composition comprising an Annexin A1 (AnxA1) N-terminal-peptide and at least one pharmaceutically acceptable excipient for use in the treatment of an ischemic condition and/or a reperfusion injury, wherein a first dosage of the AnxA1 N-terminal-peptide is administered to a human subject more than 1 hour post establishment of reperfusion.

In some embodiments, the first dosage of the AnxA1 N-terminal-peptide is administered to the human subject at least 1 hour and 15 minutes post establishment of reperfusion, such as at least 1 hour and 30 minutes post establishment of reperfusion, such as at least 1 hour and 45 minutes post establishment of reperfusion, such as at least 2 hours post establishment of reperfusion, such as at least 2 hours and 15 minutes post establishment of reperfusion, such as at least 2 hours and 30 minutes post establishment of reperfusion, such as at least 2 hours and 45 minutes post establishment of reperfusion, such as at least 3 hours post establishment of reperfusion.

In some embodiments, the first dosage of the AnxA1 N-terminal-peptide is administered to the human subject at least 2, such as at least 3, such as at least 4, such as at least 5 or such as at least 6 hours post establishment of reperfusion.

In some embodiments, the first dosage of the AnxA1 N-terminal-peptide is administered to the human subject about 1 hour and 15 minutes to about 1 hour and 30 minutes post establishment of reperfusion, such as about 1 hour and 30 minutes to about 1 hour and 45 minutes post establishment of reperfusion, such as about 1 hour and 45 minutes to about 2 hours post establishment of reperfusion, such as about 2 hours to about 2 hours and 15 minutes post establishment of reperfusion, such as about 2 hours and 15 minutes to about 2 hours and 30 minutes post establishment of reperfusion, such as about 2 hours and 30 minutes to about 2 hours and 45 minutes post establishment of reperfusion, such as about 2 hours and 45 minutes to about 3 hours post establishment of reperfusion, such as about 3 hours to about 3 hours and 30 minutes post establishment of reperfusion, such as about 3 hours and 30 minutes to about 4 hours post establishment of reperfusion, such as about 4 hours to about 4 hours and 30 minutes post establishment of reperfusion, such as about 4 hours and 30 minutes to about 5 hours post establishment of reperfusion, such as about 5 hours to about 5 hours and 30 minutes post establishment of reperfusion, such as about 5 hours and 30 minutes to about 6 hours post establishment of reperfusion.

In some embodiments, the first dosage of the AnxA1 N-terminal-peptide is administered to the human subject at least 2 hours post establishment of reperfusion.

In some embodiments, the AnxA1 N-terminal-peptide is administered to the human subject as a single dosage, such as a single daily dosage.

In some embodiments, the AnxA1 N-terminal-peptide is administered to the human subject as multiple dosages, such as 2, 3, 4 or 5 dosages.

In some embodiments, the AnxA1 N-terminal-peptide is administered to the human subject as multiple daily dosages, such as 2, 3, 4 or 5 daily dosages.

In some embodiments, the AnxA1 N-terminal-peptide is administered to the human subject as 3 daily dosages.

In some embodiments, the AnxA1 N-terminal-peptide is administered to the human subject as 3 daily dosages at a dosing interval of 1 to 6 hours;
such as at a dosing interval of about 1 to about 2 hours, such as at a dosing interval of about 2 to about 3 hours, such as at a dosing interval of about 3 to about 4 hours, such as at a dosing interval of about 4 to about 5 hours, such as at a dosing interval of about 5 to about 6 hours;
such as at a dosing interval of about 1 hour, such as at a dosing interval of about 2 hours, such as at a dosing interval of about 3 hours, such as at a dosing interval of about 4 hours, such as at a dosing interval of about 5 hours, such as at a dosing interval of about 6 hours.

In some embodiments, the AnxA1 N-terminal-peptide is administered to the human subject as 3 daily dosages at a dosing interval of about 3 hours.

In some embodiments, the AnxA1 N-terminal-peptide is administered to the human subject as 3 daily dosages at:
a. about 1 hour and 15 minutes to about 1 hour and 30 minutes post establishment of reperfusion, about 4 hour and 15 minutes to about 4 hour and 30 minutes post establishment of reperfusion and about 7 hour and 15 minutes to about 7 hour and 30 minutes post establishment of reperfusion, or
b. about 1 hour and 30 minutes to about 1 hour and 45 minutes post establishment of reperfusion, about 4 hour and 30 minutes to about 4 hour and 45 minutes post establishment of reperfusion and about 7 hour and 30 minutes to about 7 hour and 45 minutes post establishment of reperfusion, or
c. about 1 hour and 45 minutes to about 2 hours post establishment of reperfusion, about 4 hour and 45 minutes to about 5 hours post establishment of reperfusion and about 7 hour and 45 minutes to about 8 hours post establishment of reperfusion, or
d. about 2 hours to about 2 hours and 30 minutes post establishment of reperfusion, about 5 hours to about 5 hours and 30 minutes post establishment of reperfusion and about 8 hours post to about 8 hours and 30 minutes post establishment of reperfusion, or
e. about 3 hours to about 4 hours post establishment of reperfusion, about 6 hours to about 7 hours post establishment of reperfusion and 9 hours to about 10 hours post establishment of reperfusion, or
f. about 4 hours to about 5 hours post establishment of reperfusion, about 7 hours to about 8 hours post establishment of reperfusion and about 10 hours to about 11 hours post establishment of reperfusion, or
g. about 5 hours to about 6 hours post establishment of reperfusion, about 8 hours to about 9 hours post establishment of reperfusion and about 11 hours post to about 12 hours post establishment of reperfusion, or
h. about 6 hours to about 7 hours post establishment of reperfusion, about 9 hours to about 10 hours post establishment of reperfusion and about 12 hours to about 13 hours post establishment of reperfusion.

In some embodiments, the AnxA1 N-terminal-peptide is administered to the human subject as 3 daily dosages at:
a. about 1 hour and 15 minutes post establishment of reperfusion, about 4 hour and 15 minutes post establishment of reperfusion and about 7 hour and 15 minutes post establishment of reperfusion, or
b. about 1 hour and 30 minutes post establishment of reperfusion, about 4 hour and 30 minutes post establishment of reperfusion and about 7 hour and 30 minutes post establishment of reperfusion, or
c. about 1 hour and 45 minutes post establishment of reperfusion, about 4 hour and 45 minutes post establishment of reperfusion and about 7 hour and 45 minutes post establishment of reperfusion, or
d. about 2 hours post establishment of reperfusion, about 5 hours post establishment of reperfusion and about 8 hours post establishment of reperfusion, or
e. about 3 hours post establishment of reperfusion, about 6 hours post establishment of reperfusion and 9 hours post establishment of reperfusion, or
f. about 4 hours post establishment of reperfusion, about 7 hours post establishment of reperfusion and about 10 hours post establishment of reperfusion, or
g. about 5 hours post establishment of reperfusion, about 8 hours post establishment of reperfusion and about 11 hours post establishment of reperfusion, or
h. about 6 hours post establishment of reperfusion, about 9 hours post establishment of reperfusion and about 12 hours post establishment of reperfusion.

In some embodiments, the AnxA1 N-terminal-peptide is administered to the human subject about 2 hours post establishment of reperfusion, about 5 hours post establishment of reperfusion and about 8 hours post establishment of reperfusion.

In some embodiments, the AnxA1 N-terminal-peptide or pharmaceutical composition is administered as one or more intravenous (i.v.) infusions.

In some embodiments, the AnxA1 N-terminal-peptide or pharmaceutical composition is administered as a 1-60 minutes i.v. infusion, such as a 10-50 minutes i.v. infusion, such as a 20-40 minutes i.v. infusion, such as a 30 minutes i.v. infusion.

In some embodiments, the AnxA1 N-terminal-peptide or pharmaceutical composition is administered as a 30 minutes i.v. infusion.

In some embodiments, the AnxA1 N-terminal-peptide is administered to the human subject for 1 day, such as 3 daily dosages for 1 day.

In some embodiments, the AnxA1 N-terminal-peptide is administered to the human subject for 2 consecutive days, such as 3 daily dosages for 2 consecutive days.

In some embodiments, the AnxA1 N-terminal-peptide is administered to the human subject for 3 consecutive days, such as 3 daily dosages for 3 consecutive days.

In some embodiments, the AnxA1 N-terminal-peptide is administered to the human subject for 2 days, 3 days, 4 days, 5 days or 6 days, such as 3 daily dosages for 2 days, 3 daily dosages for 3 days, 3 daily dosages for 4 days, 3 daily dosages for 5 days or 3 daily dosages for 6 days.

In some embodiments, the AnxA1 N-terminal-peptide is selected from the group consisting of:

(SEQ ID NO: 2; Annexin A1 2-50)
a. AMVSEFLKQAWFIENEEQEYVQTVKSSKGGPGSAVSPYPTFNPSSD
VAA,
(SEQ ID NO: 3; Annexin A1 2-48)
b. AMVSEFLKQAWFIENEEQEYVQTVKSSKGGPGSAVSPYPTFNPSSD
V,
(SEQ ID NO: 4; Annexin A1 2-46)
c. AMVSEFLKQAWFIENEEQEYVQTVKSSKGGPGSAVSPYPTFNPSS,
(SEQ ID NO: 5; Annexin A1 2-50 V24L)
d. AMVSEFLKQAWFIENEEQEYVQTLKSSKGGPGSAVSPYPTFNPSSD
VAA,
(SEQ ID NO: 6; Annexin A1 2-48 V24L)
e. AMVSEFLKQAWFIENEEQEYVQTLKSSKGGPGSAVSPYPTFNPSSD
V,
(SEQ ID NO: 7; Annexin A1 2-46 V24L)
f. AMVSEFLKQAWFIENEEQEYVQTLKSSKGGPGSAVSPYPTFNPSS,
and
g. a functional variant of any one of SEQ ID NOs:
2-7 comprising 1 to 6 individual amino acid
substitutions.

In some embodiments, the functional variant comprises 1-3 individual amino acid substitutions, such as 1 individual amino acid substitution, 2 individual amino acid substitutions or 3 individual amino acid substitutions.

In some embodiments, the one or more individual amino acid substitutions are conservative amino acid substitutions.

In some embodiments, the functional variant is a ligand and/or agonist of one or more of Formyl Peptide Receptor 1 (FPR1), Formyl Peptide Receptor 2 (FPR2) and Formyl Peptide Receptor 3 (FPR3).

In some embodiments, the functional variant activates and/or stimulates one or more of Formyl Peptide Receptor 1 (FPR1), Formyl Peptide Receptor 2 (FPR2) and Formyl Peptide Receptor 3 (FPR3).

In some embodiments, the AnxA1 N-terminal-peptide has a C-terminal amidation.

In some embodiments, the AnxA1 N-terminal-peptide is AMVSEFLKQAWFIENEEQEYVQTLKSSKGGPGSAVSPYPTFNPSSDV (SEQ ID NO: 6; Annexin A1 2-48 V24L), optionally with a C-terminal amidation.

In some embodiments, the AnxA1 N-terminal-peptide is administered as a therapeutically effective amount.

In some embodiments, the AnxA1 N-terminal-peptide is administered as a dosage of about 15 μg/kg, about 25 μg/kg, about 50 μg/kg, about 100 μg/kg, about 150 μg/kg, about 200 μg/kg or about 400 μg/kg of the AnxA1 N-terminal-peptide.

In some embodiments, the ischemic condition and/or a reperfusion injury is a myocardial ischemic condition and/or reperfusion injury of the heart.

In some embodiments, the ischemic condition and/or a reperfusion injury is selected from the group consisting of coronary artery diseases, such as angina and myocardial infarction; stroke, heart failure; peripheral artery disease; thromboembolic disease; and venous thrombosis.

In some embodiments, the ischemic condition and/or a reperfusion injury is coronary artery disease.

In some embodiments, the condition and/or a reperfusion injury is myocardial infarction.

In some embodiments, the ischemic condition and/or a reperfusion injury is acute myocardial infarction.

In some embodiments, the ischemic condition and/or a reperfusion injury is myocardial infarction followed by reperfusion.

In some embodiments, the ischemic condition and/or a reperfusion injury is selected from the group consisting of myocardial infarction due to myocardial ischemia/reperfusion, myocardial infarction due to myocardial ischemia, myocarditis, sepsis, sepsis-induced cardiomyopathy, and sepsis-induced myocardial inflammation.

In some embodiments, the ischemic condition and/or a reperfusion injury is myocardial infarction due to myocardial ischemia or myocardial infarction due to myocardial ischemia/reperfusion.

In some embodiments, the myocardial infarction is selected from the group consisting of ST-segment elevation myocardial infarction (STEMI) and non-ST-segment elevation myocardial infarction (NSTEMI).

In some embodiments, the myocardial infarction is ST-elevation myocardial infarction (STEMI).

In some embodiments, the ischemic condition and/or a reperfusion injury results in an inflammatory immune response and/or tissue destruction.

In some embodiments, administration of the AnxA1 N-terminal-peptide reduces or prevents an inflammatory immune response in the ischemic tissue.

In some embodiments, administration of the AnxA1 N-terminal-peptide reduces necrosis caused by the ischemic condition and/or a reperfusion injury.

In some embodiments, the necrosis is reduced compared to necrosis caused by an ischemic condition and/or a reperfusion injury in a human subject not receiving any AnxA1 N-terminal-peptide administrations.

In some embodiments, administration of the AnxA1 N-terminal-peptide reduces the infarct size of an infarct caused by the ischemic condition and/or a reperfusion injury.

In some embodiments, the infarct size is reduced compared to infarct size of an infarct caused by an ischemic condition and/or a reperfusion injury in a human subject not receiving any AnxA1 N-terminal-peptide administrations.

In some embodiments, the administration of the AnxA1 N-terminal-peptide reduces myocardial infarct size of a myocardial infarct caused by the ischemic condition and/or a reperfusion injury.

In some embodiments, the myocardial infarct size is reduced compared to myocardial infarct size of a myocardial infarct caused by an ischemic condition and/or a reperfusion injury in a human subject not receiving any AnxA1 N-terminal-peptide administrations.

In some embodiments, the pharmaceutical composition is a liquid pharmaceutical composition comprising the AnxA1 N-terminal-peptide and at least one pharmaceutically acceptable excipient.

In some embodiments, the liquid pharmaceutical composition comprises 0.5-10 mg/mL of the AnxA1 N-terminal-peptide, such as 1-10 mg/mL, such as 2-9 mg/mL, such as 3-8 mg/mL, such as 4-6 mg/mL.

In some embodiments, the liquid pharmaceutical composition comprises:
i) 1-10 mg/mL of the AnxA1 N-terminal-peptide,
ii) 3-5% (w/w) of a sugar alcohol, and
iii) 0.5-1.5% (w/w) of a carbohydrate.

In some embodiments, the liquid pharmaceutical composition comprises:
i) 1-10 mg/mL of the AnxA1 N-terminal-peptide,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer, and
iii) 0.5-1.5% (w/w) of a carbohydrate selected from the group consisting of sucrose, trehalose, mannose, ribose, maltose, glucose, lactose, galactose and arabinose.

In some embodiments, the liquid pharmaceutical composition comprises:
i) 1-10 mg/mL of the AnxA1 N-terminal-peptide,
ii) 3-5% (w/w) of a sugar alcohol,
iii) 0.5-1.5% (w/w) of a carbohydrate,
iv) 0.005-0.015% (w/w) of a surfactant, and
v) 5-15 mM buffer.

In some embodiments, the liquid pharmaceutical composition has a pH of 8.2-8.4, the liquid pharmaceutical composition comprising
i) 1-10 mg/mL of the AnxA1 N-terminal-peptide,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer,
iii) 0.5-1.5% (w/w) of a carbohydrate selected from the group consisting of sucrose, trehalose, mannose, ribose, maltose, glucose, lactose, galactose and arabinose,
iv) 0.005-0.015% (w/w) of a surfactant selected from the group consisting of Polysorbate-80 and Polysorbate-20, and
v) 5-15 mM buffer selected from the group consisting of Acetate buffer, TRIS, Amine-containing buffers, Amino-acid based buffers such as lysine, hydroxy-lysine histidine and glycylglycine, non-phosphate buffers, bicarbonate buffers, HEPES and borate buffers.

In some embodiments, the liquid pharmaceutical composition is administered as a once daily i.v. infusion, such as a once daily 30 minutes i.v. infusion.

In some embodiments, the liquid pharmaceutical composition is administered as multiple daily i.v. infusions, such as 2, 3, 4 or 5 daily i.v. infusions.

In some embodiments, the once daily i.v. infusion or multiple daily i.v. infusions correspond to administration of a dosage of about 15 μg/kg, about 25 μg/kg, about 50 μg/kg, about 100 μg/kg, about 150 μg/kg, about 200 μg/kg, or about 400 μg/kg of the AnxA1 N-terminal-peptide.

In some embodiments, the once daily i.v. infusion or multiple daily i.v. infusions correspond to administration of a dosage of about 15-400 μg/kg, about 25-400 μg/kg, about 50-400 μg/kg, about 100-400 μg/kg, about 150-400 μg/kg, or about 200-400 μg/kg of the AnxA1 N-terminal-peptide

In some embodiments, the once daily i.v. infusion or multiple daily i.v. infusions correspond to administration of a dosage of about 15-400 μg/kg, about 25-400 μg/kg, about 25-200 μg/kg, about 25-150 μg/kg, about 25-100 μg/kg, or about 25-50 μg/kg of the AnxA1 N-terminal-peptide.

In some embodiments, the once daily i.v. infusion or multiple daily i.v. infusions correspond to administration of a dosage of about 25-200 μg/kg of the AnxA1 N-terminal-peptide.

In some embodiments, the liquid pharmaceutical composition is administered as 3 daily i.v. infusions.

In some embodiments, the liquid pharmaceutical composition is administered 3 times daily each as a 20-40 minutes i.v. infusion, such as each as a 30 minutes i.v. infusion.

In some embodiments, each i.v. infusion corresponds to administration of a dosage of about 25-200 μg/kg, about 25-150 μg/kg, about 25-100 μg/kg, or about 25-50 μg/kg of the AnxA1 N-terminal-peptide.

In some embodiments, the liquid pharmaceutical composition is administered by i.v. infusion with a flow rate of about 0.5 to 10 mL/min., such as of 0.5 to 10 mL/min., such as of 0.5 to 5 mL/min., such as of 0.5 to 4 mL/min., such as of 0.5 to 3 mL/min., such as of 1 to 2 mL/min., such as of about 1.5 mL/min.

In some embodiments, the pharmaceutical composition is administered by i.v. infusion with a flow rate of 1.5 mL/min.

### EXAMPLES

In the following Examples, reference to ‘Peptide 1’ is identified herein as AMVSEFLKQAWFIENEEQEYVQTLKSSKGGPGSAVSPYPTFNPSSDV (SEQ ID NO: 6 (Annexin A1 2-48 V24L)), with a C-terminal amidation.

### Example 1: Effect of Delayed Annexin A1 Peptide Dosing in a Rat Model of Acute Myocardial Infarction

### Method:

Myocardial infarction was induced by 30 minutes ischemia via obstruction of coronary perfusion by occlusion of the left coronary artery followed by 24 hours reperfusion. Sprague Dawley rats were treated by administration with Peptide 1 (30 ug/kg) three times with treatment initiation either at the time of reperfusion (T0 h, T3 h, T6 h) (Group 1), or with a delayed treatment initiation starting either at 1 hour post establishment of reperfusion (T1 h, T4 h, T7 h) (Group 2), or at 2 hours post establishment of reperfusion (T2 h, T5 h, T8 h) (Group 3).

At the end of 24 hours reperfusion period, the left coronary artery was reoccluded and Evans blue dye solution injected. Hearts were excised from the rat and stored at −20° C. before being sliced into 1 mm thick transverse sections. Heart sections were incubated with triphenyltetrazolium chloride (TTC, 1%) at 37° C. for 30 minutes to demarcate the viable and nonviable myocardium within the risk zone. Area at risk and infarcted area were determined using image software.

### Results:

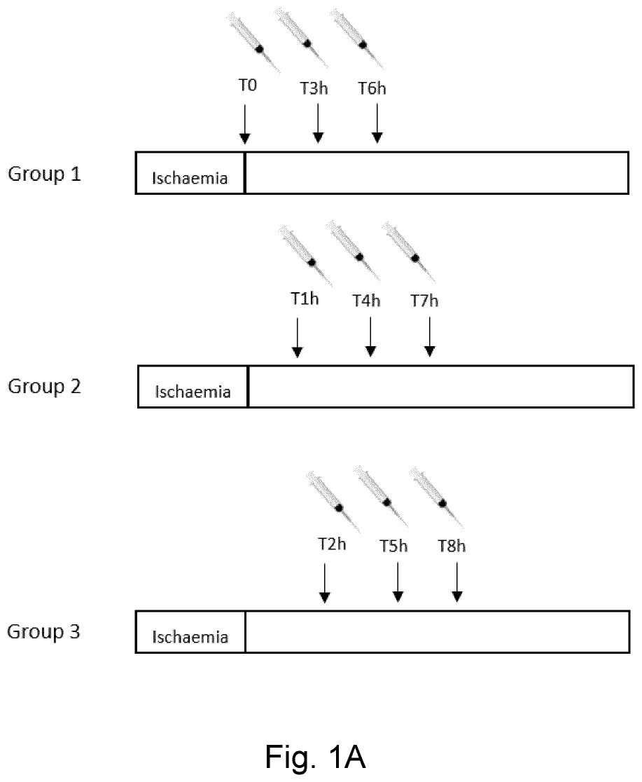

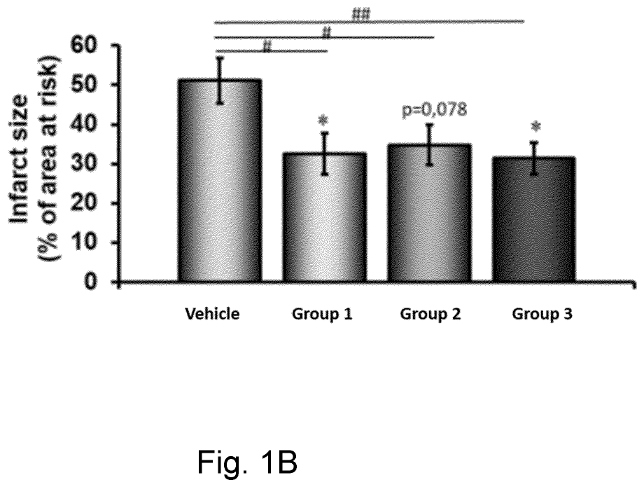

Peptide 1 significantly reduced infarct size when administered 3 times, with the first dosage given at establishment of reperfusion (reperfusion start) (Group 1). Delaying the initiation of treatment with Peptide 1 for both one and two hours reduced infarct size to a similar degree as when Peptide 1 was administered immediately at establishment of reperfusion (reperfusion start) (FIG. 1B). Thus, delaying Peptide 1 administration for at least two hours post establishment of reperfusion does not compromise the cardioprotective effects of Peptide 1.

### Example 2: Effect of Peptide 1 Treatment for 1 Day Versus 3 Days on Cardiac Function in a Rat Model of Acute Myocardial Infarction

### Method:

Myocardial infarction was induced in male Sprague Dawley rats. On day 0 rats were anaesthetized with a mixture of 2 to 2.2% isoflurane USP (Abbot Laboratories, Montreal Canada) in oxygen, and placed on a heating pad (set at 37° C.) to maintain body temperature. The animals were intubated and immediately ventilated with a mixture of 2 to 2.2% isoflurane USP (Abbot Laboratories, Montreal Canada) in oxygen by means of a positive-pressure rodent respirator (CWE model SAR-830/P) set at ≈10 mL/kg bodyweight at a frequency of 70-80 strokes/min. The pericardium was opened to expose the heart and a 5-0 wax coated braided silk suture (Sofsilk™) was placed around the left anterior descending (LAD) coronary artery, approximately 2-3 mm below the left atrium. The suture was briefly snared to verify the size and location of myocardial ischemia based on colour change and was then tied to produce a large anterolateral myocardial infarction (affecting around 45% of the LV). The suture was kept in place for 30 minutes (30 minutes of ischemia) followed by reperfusion. A group of rats receiving sham procedure was also included. Peptide 1 (or vehicle) was administrated in the tail vein three times, with the first dosage administered at establishment of reperfusion (reperfusion start), the second dosage 3 hours post establishment of reperfusion, and the third dosage 6 hours post establishment of reperfusion. Group 1 received 3×10 ug/kg for 3 days, Group 2 received 3×30 ug/kg for 3 days, Group 4 received 3×100 μg/kg for 3 days and Group 4 received 3×30 ug/kg for 1 day only. Fourteen days after ischemia, cardiac function was assessed by transthoracic echocardiography (echocardiograph model Q9, ChisonMedical Imaging Co. connected to a Chison 12.0 MHz probe).

### Results:

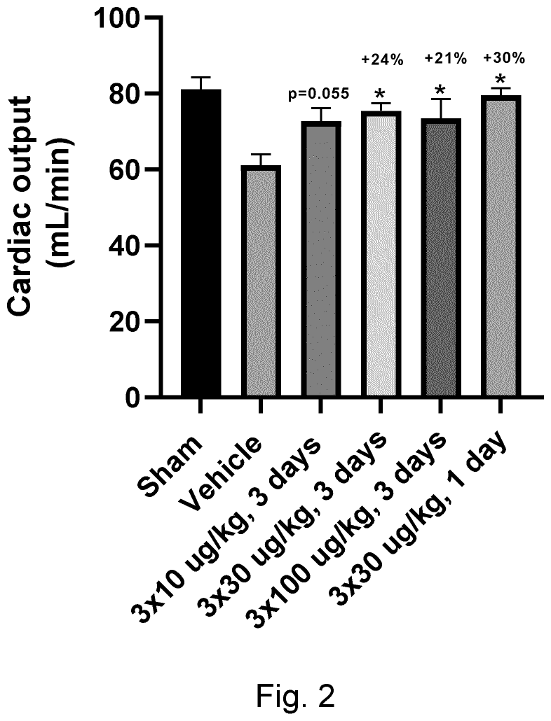

As demonstrated in FIG. 2, vehicle treatment was associated with development of impaired cardiac function with 25% reduction in cardiac output compared to sham animals. Peptide 1 administration for 1 day or 3 days significantly improved cardiac output 14 days post ischemia by 20-30% compared to vehicle treatment. Thus, administration of at least three dosages of Peptide 1 significantly improves long-term cardiac function.

### Sequence Overview

Full length Annexin A1 (Homo sapiens) has the following sequence:

>sp|P04083|ANXA1_HUMAN Annexin A1 OS = Homo sapiens
GN = ANXA1 PE = 1 SV = 2
(SEQ ID NO: 1)
MAMVSEFLKQAWFIENEEQEYVQTVKSSKGGPGSAVSPYPTFNPSSDVAAL
HKAIMVKGVDEATIIDILTKRNNAQRQQIKAAYLQETGKPLDETLKKALTG
HLEEVVLALLKTPAQFDADELRAAMKGLGTDEDTLIEILASRTNKEIRDIN
RVYREELKRDLAKDITSDTSGDFRNALLSLAKGDRSEDFGVNEDLADSDAR
ALYEAGERRKGTDVNVFNTILTTRSYPQLRRVFQKYTKYSKHDMNKVLDLE
LKGDIEKCLTAIVKCATSKPAFFAEKLHQAMKGVGTRHKALIRIMVSRSEI
DMNDIKAFYQKMYGISLCQAILDETKGDYEKILVALCGGN.
(SED ID NO: 2; Annexin A1 2-50)
AMVSEFLKQAWFIENEEQEYVQTVKSSKGGPGSAVSPYPTFNPSSDVAA
(SED ID NO: 3; Annexin A1 2-48)
AMVSEFLKQAWFIENEEQEYVQTVKSSKGGPGSAVSPYPTFNPSSDV
(SED ID NO: 4; Annexin A1 2-46)
AMVSEFLKQAWFIENEEQEYVQTVKSSKGGPGSAVSPYPTFNPSS
(SED ID NO: 5; Annexin A1 2-50 V24L)
AMVSEFLKQAWFIENEEQEYVQTLKSSKGGPGSAVSPYPTFNPSSDVAA
(SED ID NO: 6; Annexin A1 2-48 V24L)
AMVSEFLKQAWFIENEEQEYVQTLKSSKGGPGSAVSPYPTFNPSSDV
(SED ID NO: 7; Annexin A1 2-46 V24L)
AMVSEFLKQAWFIENEEQEYVQTLKSSKGGPGSAVSPYPTFNPSS
(SED ID NO: 8; Annexin A1 2-26)
AMVSEFLKQAWFIENEEQEYVQTVK,
(SED ID NO: 9; Annexin A1 2-26 V24L)
AMVSEFLKQAWFIENEEQEYVQTLK
All peptides may be C-terminally amidated (-NH₂).

1. An Annexin A1 (AnxA1) N-terminal-peptide for use in the treatment of an ischemic condition and/or a reperfusion injury, wherein a first dosage of said AnxA1 N-terminal-peptide is administered to a human subject more than 1 hour post establishment of reperfusion.

2. A pharmaceutical composition comprising an Annexin A1 (AnxA1) N-terminal-peptide and at least one pharmaceutically acceptable excipient for use in the treatment of an ischemic condition and/or a reperfusion injury, wherein a first dosage of said AnxA1 N-terminal-peptide is administered to a human subject more than 1 hour post establishment of reperfusion.

3. The AnxA1 N-terminal-peptide or pharmaceutical composition for use according to any one of the preceding claims, wherein said first dosage of said AnxA1 N-terminal-peptide is administered to the human subject at least 1 hour and 15 minutes post establishment of reperfusion, such as at least 1 hour and 30 minutes post establishment of reperfusion, such as at least 1 hour and 45 minutes post establishment of reperfusion, such as at least 2 hours post establishment of reperfusion, such as at least 2 hours and 15 minutes post establishment of reperfusion, such as at least 2 hours and 30 minutes post establishment of reperfusion, such as at least 2 hours and 45 minutes post establishment of reperfusion, such as at least 3 hours post establishment of reperfusion.

4. The AnxA1 N-terminal-peptide or pharmaceutical composition for use according to any one of the preceding claims, wherein said first dosage of said AnxA1 N-terminal-peptide is administered to the human subject at least 2 hours post establishment of reperfusion, such as at least 3, such as at least 4, such as at least 5 or such as at least 6 hours post establishment of reperfusion.

5. The AnxA1 N-terminal-peptide or pharmaceutical composition for use according to any one of the preceding claims, wherein said first dosage of said AnxA1 N-terminal-peptide is administered to the human subject about 1 hour and 15 minutes to about 1 hour and 30 minutes post establishment of reperfusion, such as about 1 hour and 30 minutes to about 1 hour and 45 minutes post establishment of reperfusion, such as about 1 hour and 45 minutes to about 2 hours post establishment of reperfusion, such as about 2 hours to about 2 hours and 15 minutes post establishment of reperfusion, such as about 2 hours and 15 minutes to about 2 hours and 30 minutes post establishment of reperfusion, such as about 2 hours and 30 minutes to about 2 hours and 45 minutes post establishment of reperfusion, such as about 2 hours and 45 minutes to about 3 hours post establishment of reperfusion, such as about 3 hours to about 3 hours and 30 minutes post establishment of reperfusion, such as about 3 hours and 30 minutes to about 4 hours post establishment of reperfusion, such as about 4 hours to about 4 hours and 30 minutes post establishment of reperfusion, such as about 4 hours and 30 minutes to about 5 hours post establishment of reperfusion, such as about 5 hours to about 5 hours and 30 minutes post establishment of reperfusion, such as about 5 hours and 30 minutes to about 6 hours post establishment of reperfusion.

6. The AnxA1 N-terminal-peptide or pharmaceutical composition for use according to any one of the preceding claims, wherein said first dosage of said AnxA1 N-terminal-peptide is administered to the human subject at least 2 hours post establishment of reperfusion.

7. The AnxA1 N-terminal-peptide or pharmaceutical composition for use according to any one of the preceding claims, wherein said AnxA1 N-terminal-peptide is administered to the human subject as a single dosage, such as a single daily dosage.

8. The AnxA1 N-terminal-peptide or pharmaceutical composition for use according to any one of the preceding claims, wherein said AnxA1 N-terminal-peptide is administered to the human subject as multiple dosages, such as 2, 3, 4 or 5 dosages.

9. The AnxA1 N-terminal-peptide or pharmaceutical composition for use according to any one of the preceding claims, wherein said AnxA1 N-terminal-peptide is administered to the human subject as multiple daily dosages, such as 2, 3, 4 or 5 daily dosages.

10. The AnxA1 N-terminal-peptide or pharmaceutical composition for use according to any one of the preceding claims, wherein said AnxA1 N-terminal-peptide is administered to the human subject as 3 daily dosages.

11. The AnxA1 N-terminal-peptide or pharmaceutical composition for use according to any one of the preceding claims, wherein said AnxA1 N-terminal-peptide is administered to the human subject as 3 daily dosages at a dosing interval of 1 to 6 hours;
such as at a dosing interval of about 1 to about 2 hours, such as at a dosing interval of about 2 to about 3 hours, such as at a dosing interval of about 3 to about 4 hours, such as at a dosing interval of about 4 to about 5 hours, such as at a dosing interval of about 5 to about 6 hours;
such as at a dosing interval of about 1 hour, such as at a dosing interval of about 2 hours, such as at a dosing interval of about 3 hours, such as at a dosing interval of about 4 hours, such as at a dosing interval of about 5 hours, such as at a dosing interval of about 6 hours.

12. The AnxA1 N-terminal-peptide or pharmaceutical composition for use according to any one of the preceding claims, wherein said AnxA1 N-terminal-peptide is administered to the human subject as 3 daily dosages at a dosing interval of about 3 hours.

13. The AnxA1 N-terminal-peptide or pharmaceutical composition for use according to any one of the preceding claims, wherein said AnxA1 N-terminal-peptide is administered to the human subject as 3 daily dosages at:
a. about 1 hour and 15 minutes to about 1 hour and 30 minutes post establishment of reperfusion, about 4 hour and 15 minutes to about 4 hour and 30 minutes post establishment of reperfusion and about 7 hour and 15 minutes to about 7 hour and 30 minutes post establishment of reperfusion, or
b. about 1 hour and 30 minutes to about 1 hour and 45 minutes post establishment of reperfusion, about 4 hour and 30 minutes to about 4 hour and 45 minutes post establishment of reperfusion and about 7 hour and 30 minutes to about 7 hour and 45 minutes post establishment of reperfusion, or
c. about 1 hour and 45 minutes to about 2 hours post establishment of reperfusion, about 4 hour and 45 minutes to about 5 hours post establishment of reperfusion and about 7 hour and 45 minutes to about 8 hours post establishment of reperfusion, or
d. about 2 hours to about 2 hours and 30 minutes post establishment of reperfusion, about 5 hours to about 5 hours and 30 minutes post establishment of reperfusion and about 8 hours to about 8 hours and 30 minutes post establishment of reperfusion, or
e. about 3 hours to about 4 hours post establishment of reperfusion, about 6 hours to about 7 hours post establishment of reperfusion and 9 hours to about 10 hours post establishment of reperfusion, or
f. about 4 hours to about 5 hours post establishment of reperfusion, about 7 hours to about 8 hours post establishment of reperfusion and about 10 hours to about 11 hours post establishment of reperfusion, or
g. about 5 hours to about 6 hours post establishment of reperfusion, about 8 hours to about 9 hours post establishment of reperfusion and about 11 hours to about 12 hours post establishment of reperfusion, or
h. about 6 hours to about 7 hours post establishment of reperfusion, about 9 hours to about 10 hours post establishment of reperfusion and about 12 hours to about 13 hours post establishment of reperfusion.

14. The AnxA1 N-terminal-peptide or pharmaceutical composition for use according to any one of the preceding claims, wherein said AnxA1 N-terminal-peptide is administered to the human subject as 3 daily dosages at:
a. about 1 hour and 15 minutes post establishment of reperfusion, about 4 hour and 15 minutes post establishment of reperfusion and about 7 hour and 15 minutes post establishment of reperfusion, or
b. about 1 hour and 30 minutes post establishment of reperfusion, about 4 hour and 30 minutes post establishment of reperfusion and about 7 hour and 30 minutes post establishment of reperfusion, or
c. about 1 hour and 45 minutes post establishment of reperfusion, about 4 hour and 45 minutes post establishment of reperfusion and about 7 hour and 45 minutes post establishment of reperfusion, or
d. about 2 hours post establishment of reperfusion, about 5 hours post establishment of reperfusion and about 8 hours post establishment of reperfusion, or
e. about 3 hours post establishment of reperfusion, about 6 hours post establishment of reperfusion and 9 hours post establishment of reperfusion, or
f. about 4 hours post establishment of reperfusion, about 7 hours post establishment of reperfusion and about 10 hours post establishment of reperfusion, or
g. about 5 hours post establishment of reperfusion, about 8 hours post establishment of reperfusion and about 11 hours post establishment of reperfusion, or
h. about 6 hours post establishment of reperfusion, about 9 hours post establishment of reperfusion and about 12 hours post establishment of reperfusion.

15. The AnxA1 N-terminal-peptide or pharmaceutical composition for use according to any of the preceding claims, wherein said AnxA1 N-terminal-peptide is administered to the human subject about 2 hours post establishment of reperfusion, about 5 hours post establishment of reperfusion and about 8 hours post establishment of reperfusion.

16. The AnxA1 N-terminal-peptide or pharmaceutical composition for use according to any one of the preceding claims, wherein said AnxA1 N-terminal-peptide or pharmaceutical composition is administered as one or more intravenous (i.v.) infusions.

17. The AnxA1 N-terminal-peptide or pharmaceutical composition for use according to any one of the preceding claims, wherein said AnxA1 N-terminal-peptide or pharmaceutical composition is administered as a 1-60 minutes i.v. infusion, such as a 10-50 minutes i.v. infusion, such as a 20-40 minutes i.v. infusion, such as a 30 minutes i.v. infusion.

18. The AnxA1 N-terminal-peptide or pharmaceutical composition for use according to any one of the preceding claims, wherein said AnxA1 N-terminal-peptide or pharmaceutical composition is administered as a 30 minutes i.v. infusion.

19. The AnxA1 N-terminal-peptide or pharmaceutical composition for use according to any one of the preceding claims, wherein said AnxA1 N-terminal-peptide is administered to the human subject for 1 day, such as 3 daily dosages for 1 day.

20. The AnxA1 N-terminal-peptide or pharmaceutical composition for use according to any one of the preceding claims, wherein said AnxA1 N-terminal-peptide is administered to the human subject for 2 consecutive days, such as 3 daily dosages for 2 consecutive days.

21. The AnxA1 N-terminal-peptide or pharmaceutical composition for use according to any one of the preceding claims, wherein said AnxA1 N-terminal-peptide is administered to the human subject for 3 consecutive days, such as 3 daily dosages for 3 consecutive days.

22. The AnxA1 N-terminal-peptide or pharmaceutical composition for use according to any one of the preceding claims, wherein said AnxA1 N-terminal-peptide is administered to the human subject for 2 days, 3 days, 4 days, 5 days or 6 days, such as 3 daily dosages for 2 days, 3 daily dosages for 3 days, 3 daily dosages for 4 days, 3 daily dosages for 5 days or 3 daily dosages for 6 days.

23. The AnxA1 N-terminal-peptide or pharmaceutical composition for use according to any one of the preceding claims, wherein said AnxA1 N-terminal-peptide is selected from the group consisting of:
(SEQ ID NO: 2; Annexin A1 2-50)
a. AMVSEFLKQAWFIENEEQEYVQTVKSSKGGPGSAVSPYPTFNPSSDV
AA,
(SEQ ID NO: 3; Annexin A1 2-48)
b. AMVSEFLKQAWFIENEEQEYVQTVKSSKGGPGSAVSPYPTFNPSSDV,
(SEQ ID NO: 4; Annexin A1 2-46)
c. AMVSEFLKQAWFIENEEQEYVQTVKSSKGGPGSAVSPYPTFNPSS,
(SEQ ID NO: 5; Annexin A1 2-50 V24L)
d. AMVSEFLKQAWFIENEEQEYVQTLKSSKGGPGSAVSPYPTFNPSSDV
AA,
(SEQ ID NO: 6; Annexin A1 2-48 V24L)
e. AMVSEFLKQAWFIENEEQEYVQTLKSSKGGPGSAVSPYPTFNPSSDV,
(SEQ ID NO: 7; Annexin A1 2-46 V24L)
f. AMVSEFLKQAWFIENEEQEYVQTLKSSKGGPGSAVSPYPTFNPSS,
and
g. a functional variant of any one of SEQ ID NOs:
2-7 comprising 1 to 6 individual amino acid
substitutions.

24. The AnxA1 N-terminal-peptide or pharmaceutical composition for use according to claim 23, wherein said functional variant comprises 1-3 individual amino acid substitutions, such as 1 individual amino acid substitution, 2 individual amino acid substitutions or 3 individual amino acid substitutions.

25. The AnxA1 N-terminal-peptide or pharmaceutical composition for use according to any of claims 23 to 24, wherein said one or more individual amino acid substitutions are conservative amino acid substitutions.

26. The AnxA1 N-terminal-peptide or pharmaceutical composition for use according to any of claims 23 to 25, wherein said functional variant is a ligand and/or agonist of one or more of Formyl Peptide Receptor 1 (FPR1), Formyl Peptide Receptor 2 (FPR2) and Formyl Peptide Receptor 3 (FPR3).

27. The AnxA1 N-terminal-peptide or pharmaceutical composition for use according to any of claims 23 to 26, wherein said functional variant activates and/or stimulates one or more of Formyl Peptide Receptor 1 (FPR1), Formyl Peptide Receptor 2 (FPR2) and Formyl Peptide Receptor 3 (FPR3).

28. The AnxA1 N-terminal-peptide or pharmaceutical composition for use according to any one of the preceding claims, wherein said AnxA1 N-terminal-peptide has a C-terminal amidation.

29. The AnxA1 N-terminal-peptide or pharmaceutical composition for use according to any one of the preceding claims, wherein said AnxA1 N-terminal-peptide is AMVSEFLKQAWFIENEEQEYVQTLKSSKGGPGSAVSPYPTFNPSSDV (SEQ ID NO: 6; Annexin A1 2-48 V24L), optionally with a C-terminal amidation.

30. The AnxA1 N-terminal-peptide or pharmaceutical composition for use according to any one of the preceding claims, wherein said AnxA1 N-terminal-peptide is administered as a therapeutically effective amount.

31. The AnxA1 N-terminal-peptide or pharmaceutical composition for use according to any one of the preceding claims, wherein said AnxA1 N-terminal-peptide is administered as a dosage of about 15 μg/kg, about 25 μg/kg, about 50 μg/kg, about 100 μg/kg, about 150 μg/kg, about 200 μg/kg or about 400 μg/kg of said AnxA1 N-terminal-peptide.

32. The AnxA1 N-terminal-peptide or pharmaceutical composition for use according to any one of the preceding claims, wherein said ischemic condition and/or a reperfusion injury is a myocardial ischemic condition and/or reperfusion injury of the heart.

33. The AnxA1 N-terminal-peptide or pharmaceutical composition for use according to any one of the preceding claims, wherein said ischemic condition and/or a reperfusion injury is selected from the group consisting of coronary artery diseases, such as angina and myocardial infarction; stroke, heart failure; peripheral artery disease; thromboembolic disease; and venous thrombosis.

34. The AnxA1 N-terminal-peptide or pharmaceutical composition for use according to any one of the preceding claims, wherein said ischemic condition and/or a reperfusion injury is coronary artery disease.

35. The AnxA1 N-terminal-peptide or pharmaceutical composition for use according to any one of the preceding claims, wherein said ischemic condition and/or a reperfusion injury is myocardial infarction.

36. The AnxA1 N-terminal-peptide or pharmaceutical composition for use according to any one of the preceding claims, wherein said ischemic condition and/or a reperfusion injury is acute myocardial infarction.

37. The AnxA1 N-terminal-peptide or pharmaceutical composition for use according to any one of the preceding claims, wherein said ischemic condition and/or a reperfusion injury is myocardial infarction followed by reperfusion.

38. The AnxA1 N-terminal-peptide or pharmaceutical composition for use according to any one of the preceding claims, wherein said ischemic condition and/or a reperfusion injury is selected from the group consisting of myocardial infarction due to myocardial ischemia/reperfusion, myocardial infarction due to myocardial ischemia, myocarditis, sepsis, sepsis-induced cardiomyopathy, and sepsis-induced myocardial inflammation.

39. The AnxA1 N-terminal-peptide or pharmaceutical composition for use according to any one of the preceding claims, wherein said ischemic condition and/or a reperfusion injury is myocardial infarction due to myocardial ischemia or myocardial infarction due to myocardial ischemia/reperfusion.

40. The AnxA1 N-terminal-peptide or pharmaceutical composition for use according to claim 35, wherein said myocardial infarction is selected from the group consisting of ST-segment elevation myocardial infarction (STEMI) and non-ST-segment elevation myocardial infarction (NSTEMI).

41. The AnxA1 N-terminal-peptide or pharmaceutical composition for use according to claim 35, wherein said myocardial infarction is ST-elevation myocardial infarction (STEMI).

42. The AnxA1 N-terminal-peptide or pharmaceutical composition for use according to any one of the preceding claims, wherein said ischemic condition and/or a reperfusion injury results in an inflammatory immune response and/or tissue destruction.

43. The AnxA1 N-terminal-peptide or pharmaceutical composition for use according to any one of the preceding claims, wherein administration of said AnxA1 N-terminal-peptide reduces or prevents an inflammatory immune response in the ischemic tissue.

44. The AnxA1 N-terminal-peptide or pharmaceutical composition for use according to any one of the preceding claims, wherein administration of said AnxA1 N-terminal-peptide reduces necrosis caused by said ischemic condition and/or a reperfusion injury.

45. The AnxA1 N-terminal-peptide or pharmaceutical composition for use according to claim 44, wherein the necrosis is reduced compared to necrosis caused by an ischemic condition and/or a reperfusion injury in a human subject not receiving any AnxA1 N-terminal-peptide administrations.

46. The AnxA1 N-terminal-peptide or pharmaceutical composition for use according to any one of the preceding claims, wherein administration of said AnxA1 N-terminal-peptide reduces the infarct size of an infarct caused by said ischemic condition and/or a reperfusion injury.

47. The AnxA1 N-terminal-peptide or pharmaceutical composition for use according to claim 46, wherein the infarct size is reduced compared to infarct size of an infarct caused by an ischemic condition and/or a reperfusion injury in a human subject not receiving any AnxA1 N-terminal-peptide administrations.

48. The AnxA1 N-terminal-peptide or pharmaceutical composition for use according to any one of the preceding claims, wherein administration of said AnxA1 N-terminal-peptide reduces myocardial infarct size of a myocardial infarct caused by said ischemic condition and/or a reperfusion injury.

49. The AnxA1 N-terminal-peptide or pharmaceutical composition for use according to claim 48, wherein the myocardial infarct size is reduced compared to myocardial infarct size of a myocardial infarct caused by an ischemic condition and/or a reperfusion injury in a human subject not receiving any AnxA1 N-terminal-peptide administrations.

50. The pharmaceutical composition for use according to any one of the preceding claims, wherein said pharmaceutical composition is a liquid pharmaceutical composition comprising said AnxA1 N-terminal-peptide and at least one pharmaceutically acceptable excipient.

51. The liquid pharmaceutical composition for use according to any one of the preceding claims, wherein said liquid pharmaceutical composition comprises 0.5-10 mg/mL of said AnxA1 N-terminal-peptide, such as 1-10 mg/mL, such as 2-9 mg/mL, such as 3-8 mg/mL, such as 4-6 mg/mL.

52. The liquid pharmaceutical composition for use according to any one of the preceding claims, said liquid pharmaceutical composition comprising:
i) 1-10 mg/mL of said AnxA1 N-terminal-peptide,
ii) 3-5% (w/w) of a sugar alcohol, and
iii) 0.5-1.5% (w/w) of a carbohydrate.

53. The liquid pharmaceutical composition for use according to any one of the preceding claims, said liquid pharmaceutical composition comprising:
i) 1-10 mg/mL of said AnxA1 N-terminal-peptide,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer, and
iii) 0.5-1.5% (w/w) of a carbohydrate selected from the group consisting of sucrose, trehalose, mannose, ribose, maltose, glucose, lactose, galactose and arabinose.

54. The liquid pharmaceutical composition for use according to any one of the preceding claims, said liquid pharmaceutical composition comprising:
i) 1-10 mg/mL of said AnxA1 N-terminal-peptide,
ii) 3-5% (w/w) of a sugar alcohol,
iii) 0.5-1.5% (w/w) of a carbohydrate,
iv) 0.005-0.015% (w/w) of a surfactant, and
v) 5-15 mM buffer.

55. The liquid pharmaceutical composition for use according to any one of the preceding claims, wherein said liquid pharmaceutical composition has a pH of 8.2-8.4, said liquid pharmaceutical composition comprising
i) 1-10 mg/mL of said AnxA1 N-terminal-peptide,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer,
iii) 0.5-1.5% (w/w) of a carbohydrate selected from the group consisting of sucrose, trehalose, mannose, ribose, maltose, glucose, lactose, galactose and arabinose,
iv) 0.005-0.015% (w/w) of a surfactant selected from the group consisting of Polysorbate-80 and Polysorbate-20, and
v) 5-15 mM buffer selected from the group consisting of Acetate buffer, TRIS, Amine-containing buffers, Amino-acid based buffers such as lysine, hydroxy-lysine histidine and glycylglycine, non-phosphate buffers, bicarbonate buffers, HEPES and borate buffers.

56. The liquid pharmaceutical composition for use according to any one of the preceding claims, wherein said liquid pharmaceutical composition is administered as a once daily i.v. infusion, such as a once daily 30 minutes i.v. infusion.

57. The liquid pharmaceutical composition for use according to any one of the preceding claims, wherein said liquid pharmaceutical composition is administered as multiple daily i.v. infusions, such as 2, 3, 4 or 5 daily i.v. infusions.

58. The liquid pharmaceutical composition for use according to any one of the preceding claims, wherein the once daily i.v. infusion or multiple daily i.v. infusions correspond to administration of a dosage of about 15 μg/kg, about 25 μg/kg, about 50 μg/kg, about 100 μg/kg, about 150 μg/kg, about 200 μg/kg, or about 400 μg/kg of said AnxA1 N-terminal-peptide.

59. The liquid pharmaceutical composition for use according to any one of the preceding claims, wherein the once daily i.v. infusion or multiple daily i.v. infusions correspond to administration of a dosage of about 15-400 μg/kg, about 25-400 μg/kg, about 50-400 μg/kg, about 100-400 μg/kg, about 150-400 μg/kg, or about 200-400 μg/kg of said AnxA1 N-terminal-peptide

60. The liquid pharmaceutical composition for use according to any one of the preceding claims, wherein the once daily i.v. infusion or multiple daily i.v. infusions correspond to administration of a dosage of about 15-400 μg/kg, about 25-400 μg/kg, about 25-200 μg/kg, about 25-150 μg/kg, about 25-100 μg/kg, or about 25-50 μg/kg of said AnxA1 N-terminal-peptide.

61. The liquid pharmaceutical composition for use according to any one of the preceding claims, wherein the once daily i.v. infusion or multiple daily i.v. infusions correspond to administration of a dosage of about 25-200 μg/kg of said AnxA1 N-terminal-peptide.

62. The liquid pharmaceutical composition for use according to any one of the preceding claims, wherein said liquid pharmaceutical composition is administered as 3 daily i.v. infusions.

63. The liquid pharmaceutical composition for use according to any one of the preceding claims, wherein said liquid pharmaceutical composition is administered 3 times daily each as a 20-40 minutes i.v. infusion, such as each as a 30 minutes i.v. infusion.

64. The liquid pharmaceutical composition for use according to any one of the preceding claims, wherein each i.v. infusion corresponds to administration of a dosage of about 25-200 μg/kg, about 25-150 μg/kg, about 25-100 μg/kg, or about 25-50 μg/kg of said AnxA1 N-terminal-peptide.

65. The liquid pharmaceutical composition for use according to any one of the preceding claims, wherein said liquid pharmaceutical composition is administered by i.v. infusion with a flow rate of about 0.5 to 10 mL/min., such as of 0.5 to 10 mL/min., such as of 0.5 to 5 mL/min., such as of 0.5 to 4 mL/min., such as of 0.5 to 3 mL/min., such as of 1 to 2 mL/min., such as of about 1.5 mL/min.

66. The liquid pharmaceutical composition for use according to any one of the preceding claims, wherein said liquid pharmaceutical composition is administered by i.v. infusion with a flow rate of 1.5 mL/min.
